(19) United States
(12) Patent Application Publication
(10) Pub. No.: US 2016/0041615 A1
(43) Pub. Date: Feb. 11, 2016

(54) INFORMATION PROCESSING APPARATUS, FOCUS DETECTION METHOD, AND INFORMATION PROCESSING SYSTEM

(71) Applicant: FUJITSU LIMITED, Kawasaki-shi (JP)

(72) Inventor: Katsuhiko IKEDA, Hino (JP)

(21) Appl. No.: 14/747,555

(22) Filed: Jun. 23, 2015

(30) Foreign Application Priority Data
Aug. 5, 2014 (JP) 2014-159866

(51) Int. Cl. G06F 3/01; A61B 3/12; G06T 1/00; A61B 3/103; G02B 27/01; G02B 27/00; A61B 3/15; A61B 3/00

(52) U.S. Cl. CPC G06F 3/013; A61B 3/156; A61B 3/12; A61B 3/0025; A61B 3/103; G02B 27/0172; G02B 27/0093; G06T 1/00; G02B 2027/0178; G02B 2027/0138; G02B 2027/014

(57) ABSTRACT

An information processing apparatus includes: a shutter configured to block extraneous light which enters into an eye; an irradiation unit configured to irradiate a marker to the eye by infrared light; a photographing unit configured to photograph the marker which is projected onto a fundus of the eye to be examined when the shutter is closed; and a processing unit configured to detect a focus based on an image of the marker photographed by the photographing unit.

### CROSS-REFERENCE TO RELATED APPLICATION

This application is based upon and claims the benefit of priority from the prior Japanese Patent Application No. 2014-159866 filed on Aug. 5, 2014, the entire contents of which are incorporated herein by reference.

### FIELD

The embodiments discussed herein are related to an information processing apparatus, a focus detection method, and an information processing system.

### BACKGROUND

A see-through type Head Mounted Display (HMD) device projects a display image onto a half mirror which takes a portion of a field of view and superimposes a landscape of an external world and the display image to be displayed to the user.

Related techniques are disclosed in, for example, Japanese Laid-Open Patent Publication No. 2005-208625, Japanese Laid-Open Patent Publication No. 2010-134051, and Japanese Laid-Open Patent Publication No. 09-274144.

### SUMMARY

According to one aspect of the embodiments, an information processing apparatus includes: a shutter configured to block extraneous light which enters into an eye; an irradiation unit configured to irradiate a marker to the eye by infrared light; a photographing unit configured to photograph the marker which is projected onto a fundus of the eye to be examined when the shutter is closed; and a processing unit configured to detect a focus based on an image of the marker photographed by the photographing unit.

The object and advantages of the invention will be realized and attained by means of the elements and combinations particularly pointed out in the claims. It is to be understood that both the foregoing general description and the following detailed description are exemplary and explanatory and are not restrictive of the invention, as claimed.

### BRIEF DESCRIPTION OF DRAWINGS

FIG. 1 is a diagram illustrating an example of measurement of a refractive power of eye measured by an Auto Refracto-Keratometer;

FIG. 2 is a diagram illustrating another example of measurement of a refractive power of eye;

FIG. 3A illustrates an example of a perspective view of an HMD device;

FIG. 3B illustrates an example of a top view of an HMD device;

FIG. 4 illustrates an example of a configuration of an HMD device;

FIG. 5A and FIG. 5B illustrate an example of a photographed image;

FIG. 6 illustrates an example of an HMD device when carrying out a focus measurement;

FIG. 7A, FIG. 7B, and FIG. 7C illustrate an example of a marker;

FIG. 8 illustrates an example of an HMD device in an image information display phase;

FIG. 9 illustrates an example of a control process of an HMD device;

FIG. 10 illustrates an example of a control process; and

FIG. 11A and FIG. 11B illustrate an example of a display image.

### DESCRIPTION OF EMBODIMENTS

An HMD device photographs the pupil of a user and analyzes the photographed pupil image to detect a line of sight of the user. For example, the HMD device detects a direction along which the user gazes. The display image to be superimposed on the background is controlled according to the line of sight of the user.

For example, a menu including a plurality of alternatives as display images to be superimposed on the background and so on is displayed. In a system in which a selection and confirmation of the display image is made by detecting an alternative to which the line of sight of the user is directed, when, for example, the user gazes his eye on the display image to be superimposed on the background, it is considered that the user intends to focus his eye on the display image and perform an operation on the display image, such as the selection of menu. For example, when the user does not focus his eye on the display image, for example, simply views a background, it is considered that the user does not intend to perform an operation on the display image, such as the selection of menu.

In such a system, when a control is made based only on information of the line of sight of the user, for example, information indicating the direction that the user gazes, an unintended operation of the user may be performed to impair an operability of the user.

In a case where the pupil of the user is photographed and the photographed pupil image is analyzed so as to detect the line of sight of the user, information about a position on which the user focuses his eyes, for example, information of a depth direction may not be obtained.

A system may be provided which uses the information about a position on which the user focuses his eyes, for example, information of a depth direction.

The information of change in a visual observation distance of eyes which visually observes an external world may be acquired to control a viewing distance of an image according to the visual observation distance.

Infrared rays forming a certain image may be emitted to a retina to capture the certain image formed on the retina such that the focus control is performed according to the state of the captured certain image.

A gaze distance which is a distance to a position being gazed with the eyes of an observer may be detected to control the display of an imaginary image to be superimposed based on gaze distance.

In the control performed using information of the focus of user, when the focus is not detected or erroneously detected, the operation not intended by the user is performed and thus, an accurate focus detection may be needed.

In a case where a marker (measurement indicator) by infrared light is irradiated to an eye to be examined to measure the refractive power of the eye to detect a focus, when extraneous light containing strong infrared light enters the eye to be examined, the extraneous light may become disturbance (noise) with respect to the marker. Therefore, the refractive power may not be accurately measured and the focus may not be accurately detected.

A measurement of the refractive power of eye by the Auto Refracto-Keratometer may be utilized in the HMD device.

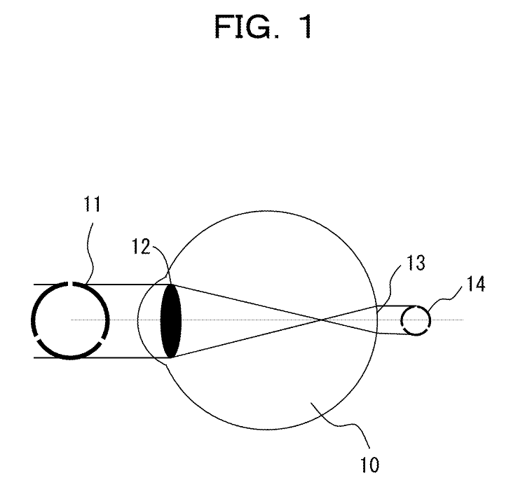

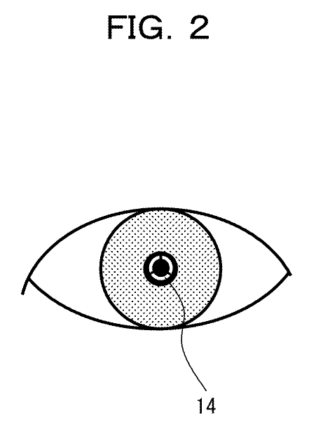

The Auto Refracto-Keratometer may be a medical instrument to measure, for example, a corneal refraction or a corneal curvature. FIG. 1 and FIG. 2 illustrate an example of measurement of a refractive power of eye by the Auto Refracto-Keratometer.

In FIG. 1, a cross-section view of an eye to be examined at the time of irradiation of a marker (measurement indicator) 11 is illustrated. In FIG. 2, an image of an eye to be examined 10 photographed by a CCD image sensor is illustrated.

In the measurement of the refractive power of the eye by the Auto Refracto-Keratometer, the marker (measurement indicator) 11 is projected onto the eye to be examined 10 by infrared light. When the user varies a focus, the shape of a crystalline lens 12 varies and a magnitude of an image of a marker 14 to be formed on a fundus 13 also varies according to the eye refractivity. The Auto Refracto-Keratometer photographs the marker 14 projected to the fundus 13 by the CCD image sensor and detects the magnitude of the image of the marker 14 generated in the fundus 13 to measure a distance to the point on which the user focuses his eyes.

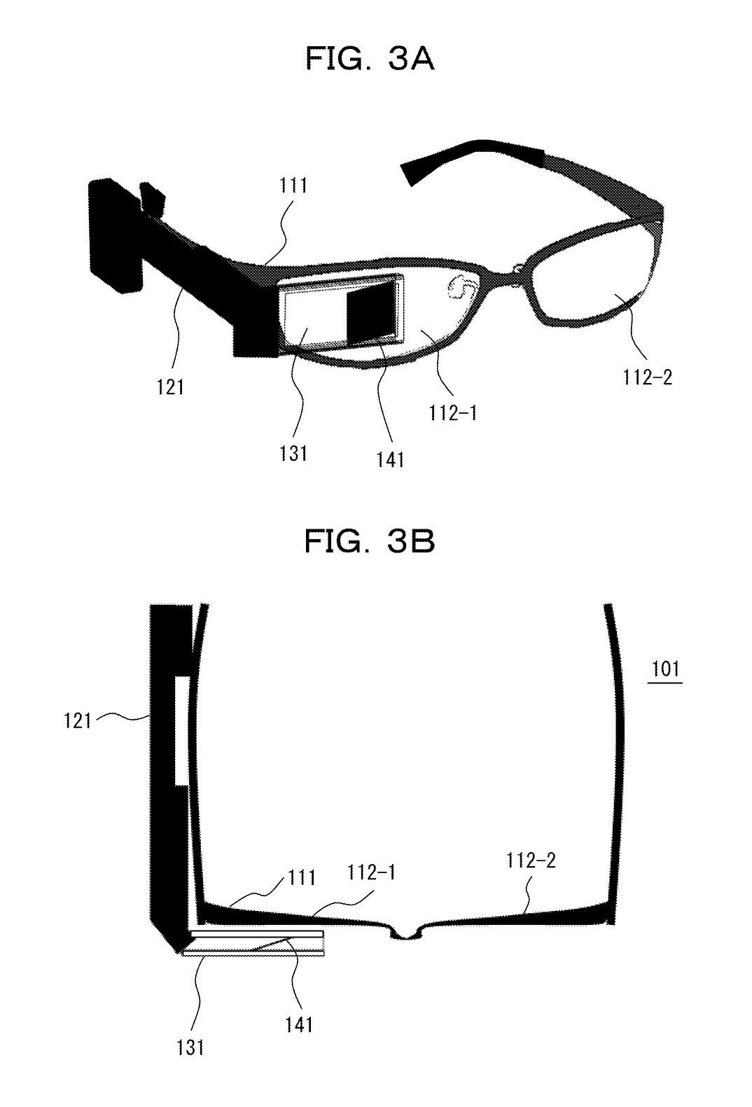

FIG. 3A illustrates an example of a perspective view of an HMD device. FIG. 3B illustrates an example of a top view of an HMD device.

An HMD device 101 includes a frame 111, a case 121, a shutter 131, and a half mirror 141.

The HMD device 101 may be used by wearing it on the head of the user. The HMD device 101 may be a see-through HMD device capable of seeing an external world through the half mirror 141. An information processing apparatus, for example, a computer may be an example of the HMD device 101.

For example, the right eye of the user may be a target eye to be examined for which the line of sight and the focus are to be detected.

The frame 111 may be an eyeglass shaped supporting frame and is capable of being worn on the head of the user. A lens portion 112-i (i=1, 2) of the frame 111 may be an example of a light entrance part. The lens portion 112-i may be an opening.

When the user wears the HMD device 101 on his head, the lens portion 112-1 and the lens portion 112-2 are located in front of the right eye and the left eye of the user, respectively.

The case 121 includes, for example, a processing device that performs various processings and a battery that supplies power to the HMD device 101. The case 121 performs a detection of a line of sight or a focus, a control of a display image, a control of the shutter 131 and so on. The case 121 is attached to a temple part of the frame 111.

The shutter 131 is disposed between the half mirror 141 and the external world. The shutter 131 is attached to the frame 111 to be disposed in front of the right eye of the user when the HMD device 101 is worn by the user. When the shutter 131 is closed, light (e.g., extraneous light) directing toward the right eye from the external world is blocked. When the shutter 131 is open, since the extraneous light transmits the shutter 131, the right eye of the user may view the external world through the lens portion 112-1 and the half mirror 141. The shutter 131 may be either a mechanical shutter or an electrical shutter using the liquid crystal technology.

The half mirror 141 is disposed between the lens portion 112-1 and the shutter 131 of the frame 111. The half mirror 141 is attached to the frame 111 to be disposed in front of the right eye of the user when the HMD device 101 is worn by the user. The half mirror 141 reflects at least a portion of light irradiated from the case 121 and allows the extraneous light to transmit. The light reflected by the half mirror 141 enters the right eye of the user. The user may view the display image projected to the half mirror 141 by being superimposed on the scene of the external world. The half mirror 141 may be a beam splitter or an optical system such as a prism. The half mirror 141 may be an example of a display unit.

When the user wears the HMD device 101, the lens portion 112-1, the half mirror 141, and the shutter 131 are disposed in this order, in front of the right eye of the user, in a direction directing from the right eye toward the external world. Accordingly, when the shutter 131 is closed, the extraneous light that enters the right eye by passing through the half mirror 141 and the lens portion 121-1 is blocked.

The shutter 131 and the half mirror 141 may be disposed in front of either the right eye or the left eye of the user and otherwise, in front of both the right eye and left eye.

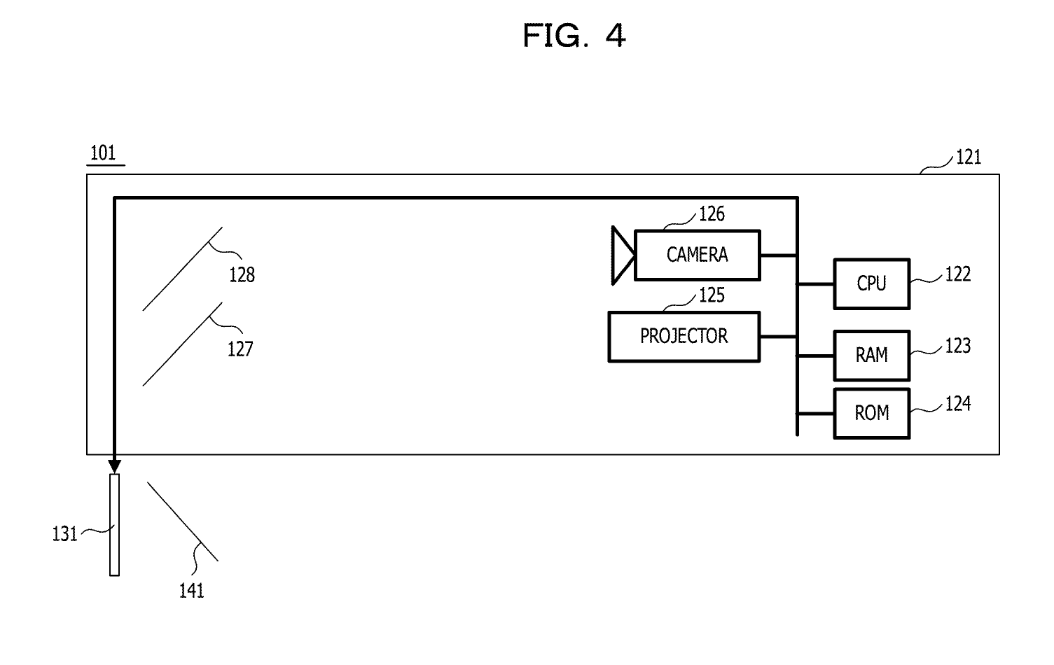

FIG. 4 illustrates an example of a configuration of an HMD device. In FIG. 4, descriptions on the frame 111 may be omitted.

The case 121 includes a central processing unit (CPU) 122, a random access memory (RAM) 123, a read only memory (ROM) 124, a projector 125, a camera 126, and half mirrors 127 and 128.

The CPU 122 may be a processing device to perform various processings. The CPU 122 reads a program or data stored in the ROM 124 into the RAM 123 and executes a control process. The CPU 122 performs a control of the projector 125, the camera 126, and the shutter 131. The CPU 122 analyzes the image photographed by the camera 125 to detect the line of sight and the focus of the user.

The RAM 123 may be a storage device to temporarily store data. The RAM 123 stores a program or data used by the CPU 122.

The ROM 124 may be a storage device to store data. The ROM 124 maintains the data even when the power is not supplied. The ROM 124 may be a flash ROM in which the stored data may be rewritten. The ROM 124 stores the program or data which is used by the CPU.

The RAM 123 and ROM 124 may be an example of the storage device. The projector 125 irradiates a measurement indicator, for example, a marker by infrared ray. For example, a shape of the marker may be circular. The projector 125 irradiates the display image to be displayed by being superimposed on the external world, for example, the background. The marker and the display image are reflected by the half mirror 127 and the half mirror 141, and enter the eye to be examined.

A projector (measurement light source) irradiating the marker and another projector irradiating the display image may be individual devices. The projector 125 may be an example of an irradiation unit.

The camera 126 photographs the eye to be examined through the half mirror 141 and the half mirror 128.

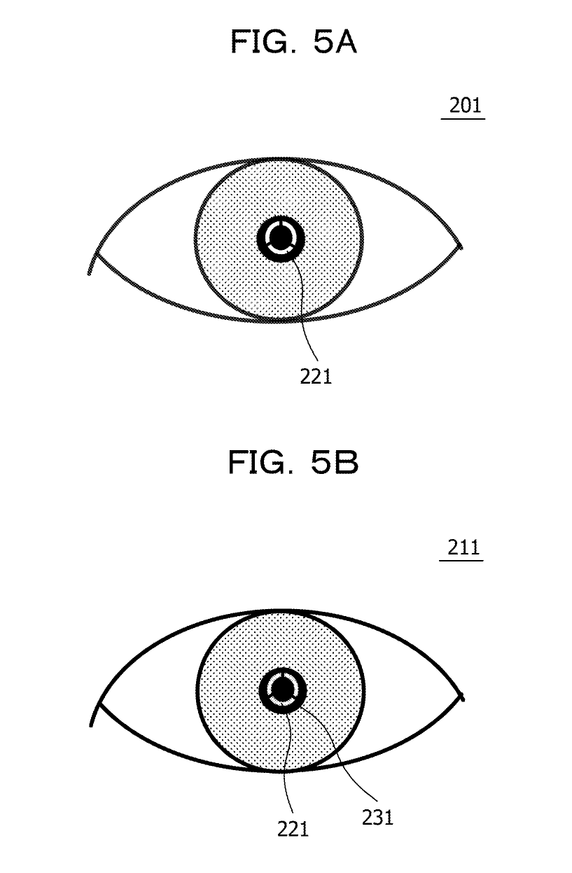

FIG. 5A and FIG. 5B illustrate an example of a photographed image. The camera 126 photographs the marker projected to a fundus of the eye to be examined. For example, the camera 126 photographs the eye to be examined by adjusting the focus to the marker. Therefore, a photographed image 201 as illustrated in FIG. 5A is obtained. Since the focus is adjusted to the marker 221 projected to the fundus in the photographed image 201 of FIG. 5A, the marker 221 has been clearly photographed. Since the shutter 131 is closed at the time when the marker 221 is photographed, the marker 221 is clearly photographed. The CPU 122 detects the magnitude of the marker 221 from the photographed image 201 and detects the focus of the user from the magnitude of the marker 221.

The camera 126 photographs the pupil of the eye (iris). For example, the camera 126 adjusts a focus to the pupil to photograph the eye to be examined. Therefore, a photographed image 211 as illustrated in FIG. 5B is obtained. Since the focus is adjusted to the pupil in the photographed image 211 of FIG. 5B, the pupil 231 has been clearly photographed. The CPU 122 detects a position of the pupil 231 from the photographed image 211 and detects a position of the line of sight, for example, a direction (angle) of the line of sight, of the user from the position of the pupil 231.

The camera 126 may be an example of a photographing unit. The half mirror 127 reflects the light irradiated from the projector 125 to the half mirror 141. The light reflected by the half mirror 127 is reflected at the half mirror 141 and enters the eye to be examined.

The half mirror 128 reflects the image of the eye to be examined projected to the half mirror 141 to the camera 126.

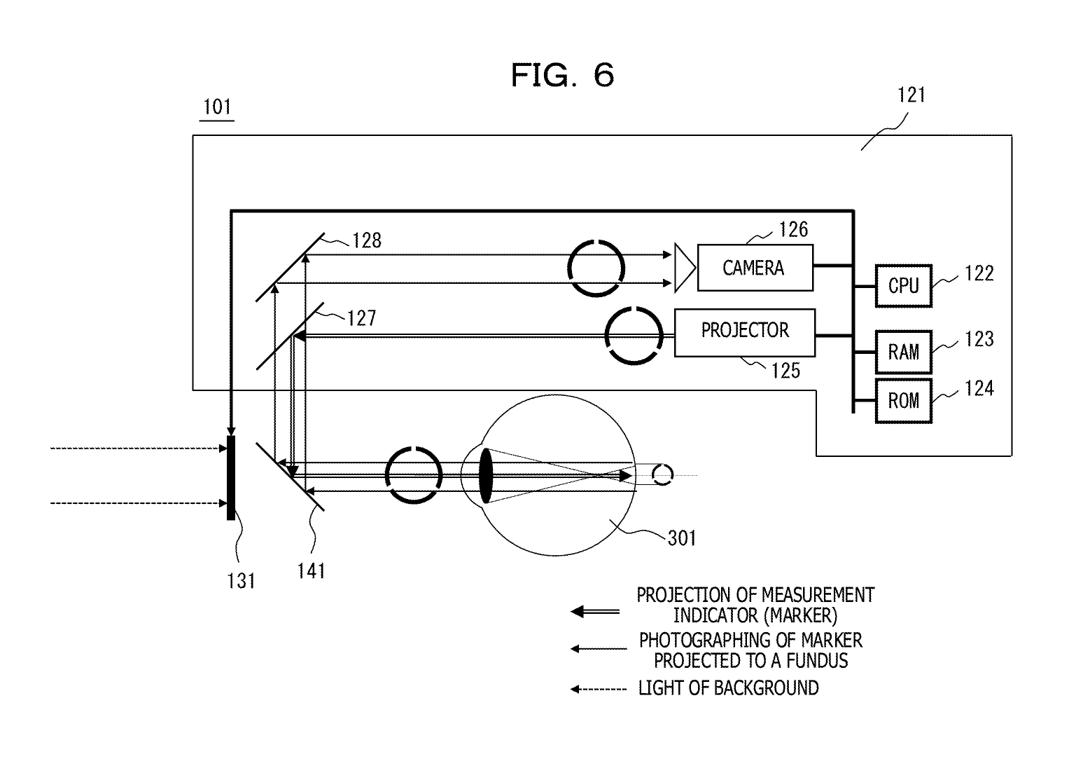

FIG. 6 illustrates an example of an HMD device when carrying out a focus measurement. In the line of sight and focus measurement phase, the CPU 122 closes the shutter 131 to block the extraneous light (light of background) directing toward the eye to be examined 301.

The projector 125 irradiates the marker by infrared ray. The marker is reflected from the half mirror 127 and the half mirror 141, and enters the eye to be examined 301. Therefore, the marker is projected to the fundus of the eye to be examined 301.

The camera 126 photographs the marker projected to the fundus of the eye to be examined 301 and the CPU 122 stores the photographed image 201 in the RAM 123. The camera 126 photographs the pupil to be examined and stores the photographed image 211 in the RAM 123. Photographing of the opening may be performed in an image information display phase. The camera 126 photographs the images of the marker and the pupil entered by being reflected from the half mirror 141 and the half mirror 128.

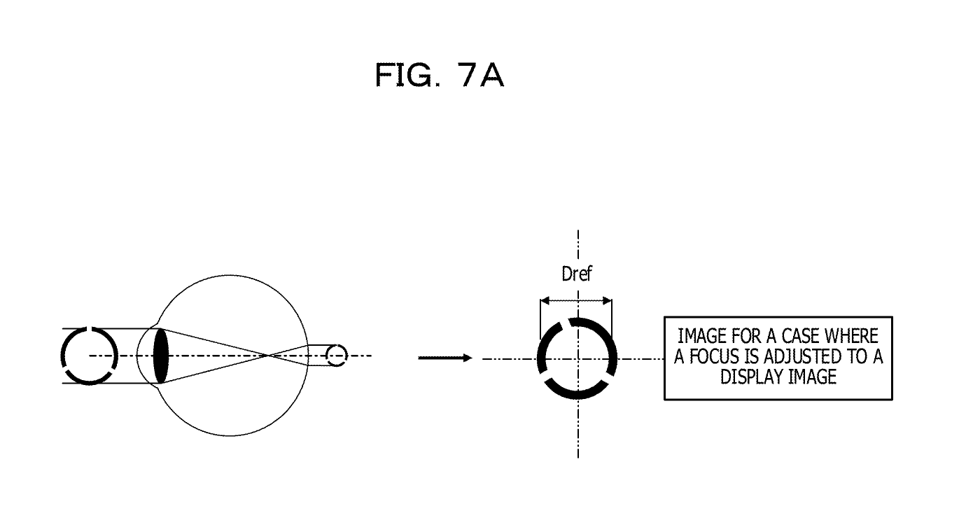

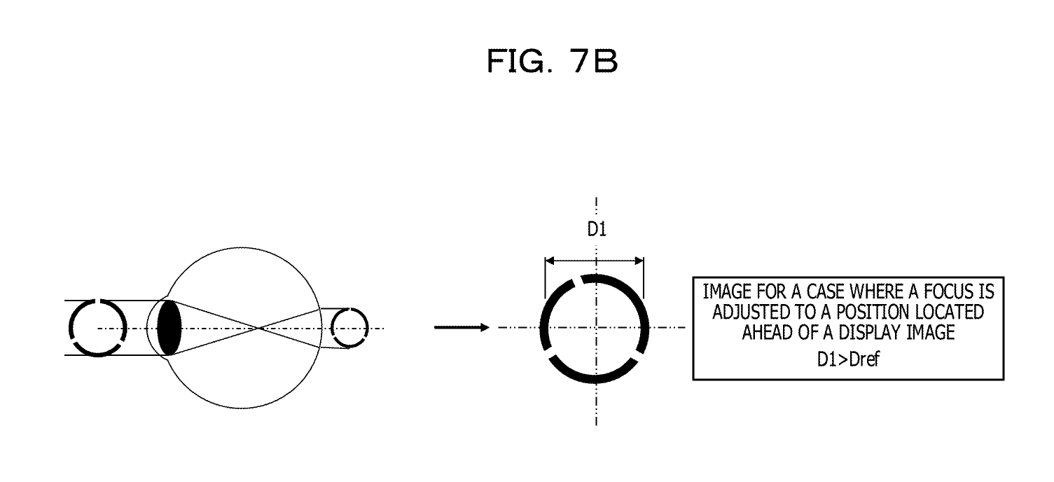

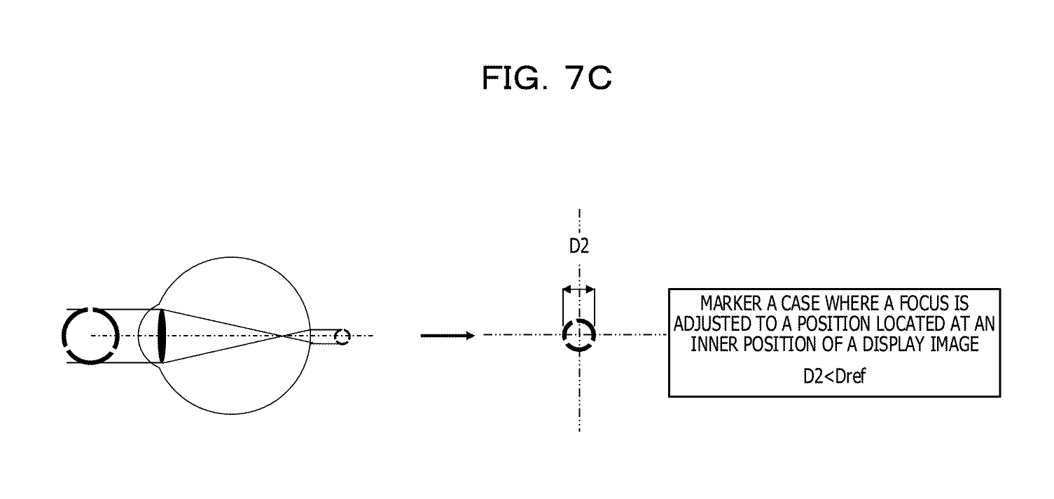

FIG. 7A, FIG. 7B, and FIG. 7C illustrate an example of a marker. In FIG. 7A, a marker for a case where the focus is adjusted to the display image is displayed.

It is assumed that, for example, the size (diameter) of the marker projected to the fundus at the time when the user adjusts the focus to the display image projected to the half mirror 141 is a reference value “Dref”.

The reference value “Dref” is measured before the control process is performed. The reference value “Dref” may be measured by the following processings.

The CPU 122 causes the projector 125 to irradiate a suitable display image. The user gazes at the display image projected to the half mirror 141. Accordingly, the focus of the user is adjusted to the display image.

The CPU 122 causes the shutter 131 to be closed and the projector 125 irradiates the marker by infrared ray instead of the display image. The camera 126 photographs the marker projected to the fundus of the eye to be examined.

The CPU 122 measures the size (diameter) of the marker from the photographed image and stores the measured value in the RAM 123 or the ROM 124 as the reference value of “Dref”.

In FIG. 7B, the marker is displayed for a case where the focus is adjusted to a position located ahead of the display image.

When the user adjusts the focus to the position located ahead of the display image, a size “D1” of the marker becomes larger than the reference value of “Dref”.

In FIG. 7C, the marker is displayed for a case where the focus is adjusted to a position located at an inner position of the display image.

When the user adjusts the focus to the position located at the inner position of the display image, the size “D2” of the marker becomes smaller than the reference value of “Dref”.

As described above, according to the position of the focus, the size of the marker projected to the fundus varies. Therefore, the CPU 122 measures the size of the marker from the image 201 of the photographed marker and compares a measured result with the reference value of “Dref” to detect the focus of the user.

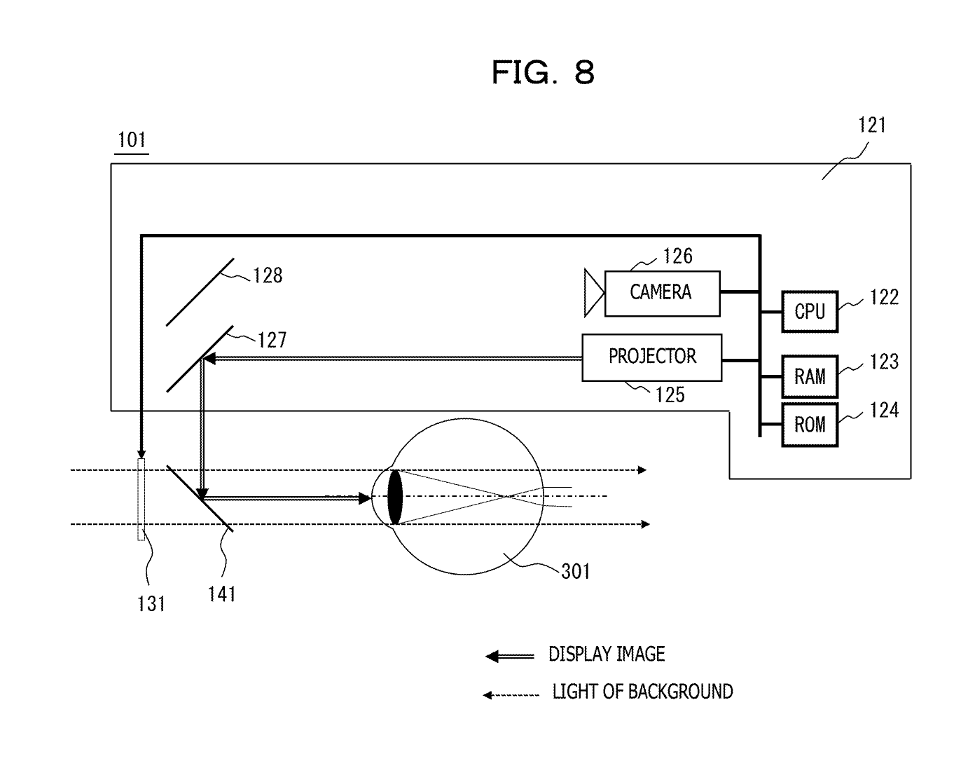

FIG. 8 illustrates an example of an HMD device in an image information display phase. In the image information display phase in which a display image is displayed, the CPU 122 causes the shutter 131 to be open to allow the extraneous light (light of background) to pass through. Therefore, the user may view the external world (background) through the half mirror 141.

The projector 125 irradiates the display image. The irradiated display image is reflected from the half mirror 127 and the half mirror 141, and enters the eye to be examined 301. Therefore, the user may view the display image by being superimposed on the external world (background) by the half mirror 141.

The CPU 122 detects the focus based on the image 201 of the marker photographed in the line of sight and focus measurement phase. The CPU 122 detects the line of sight based on the image 211 of the pupil photographed in the line of sight and focus measurement phase. The CPU 122 controls an operation corresponded to the display image based on the detected results of the line of sight and the focus.

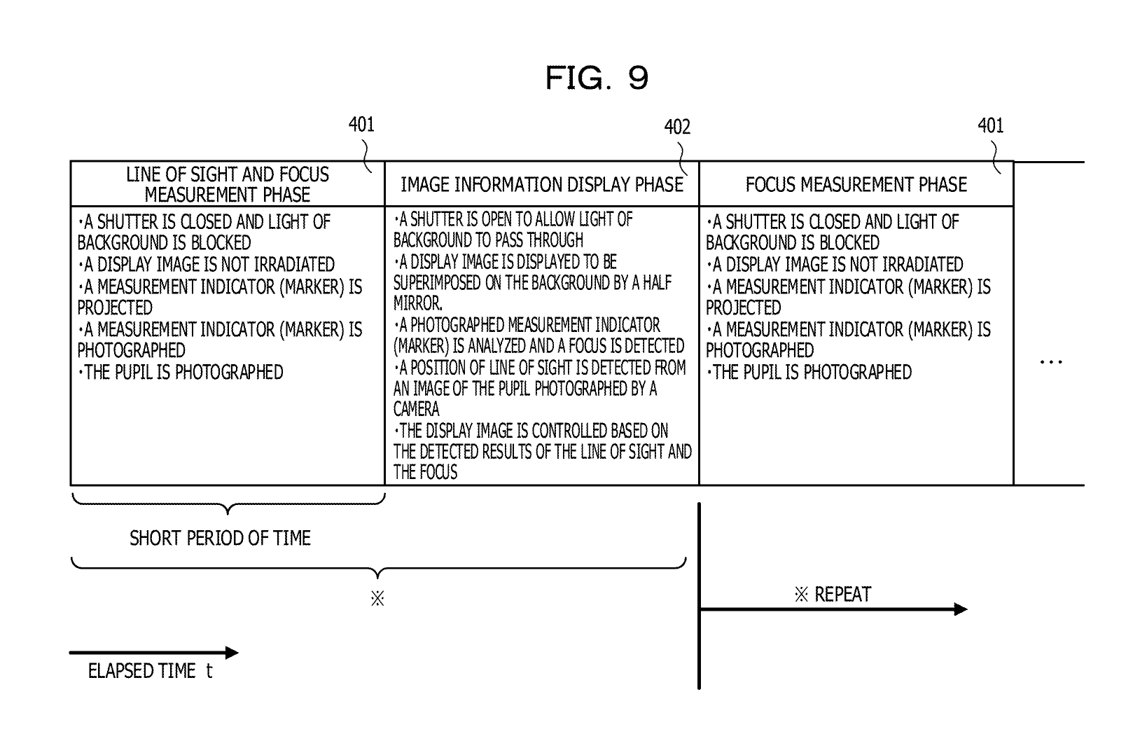

FIG. 9 illustrates an example of a control process of an HMD device. The CPU 122 repeatedly executes a line of sight and focus measurement phase 401 and an image information display phase 402.

In the line of sight and focus measurement phase 401, the shutter 13 is closed and the extraneous light is blocked. The display image is not irradiated (displayed) from the projector 125. The marker by the infrared ray is irradiated (displayed) from the projector 125. The marker projected to the fundus of the eye to be examined is photographed. The pupil of the eye to be examined is photographed. The pupil may be photographed in the image information display phase 402.

A time period of the line of sight and focus measurement phase 401, for example, a time period during which the shutter 131 is closed, may be a short time period, during which the user is not able to sense a state that the shutter 131 is being closed, for example, 5 milliseconds to 10 milliseconds. The line of sight and focus measurement phase 401 may be regularly executed.

In the image information display phase 402, the shutter 131 is open to allow the extraneous light to pass through. The display image is irradiated from the projector 125 and displayed with being superimposed on the external world (background) by the half mirror 141. The marker by infrared ray is not irradiated from the projector 125. The focus is detected based on the image of the photographed marker. The line of sight is detected based on the image of the photographed pupil. The display image is controlled based on the detected results of the line of sight and the focus.

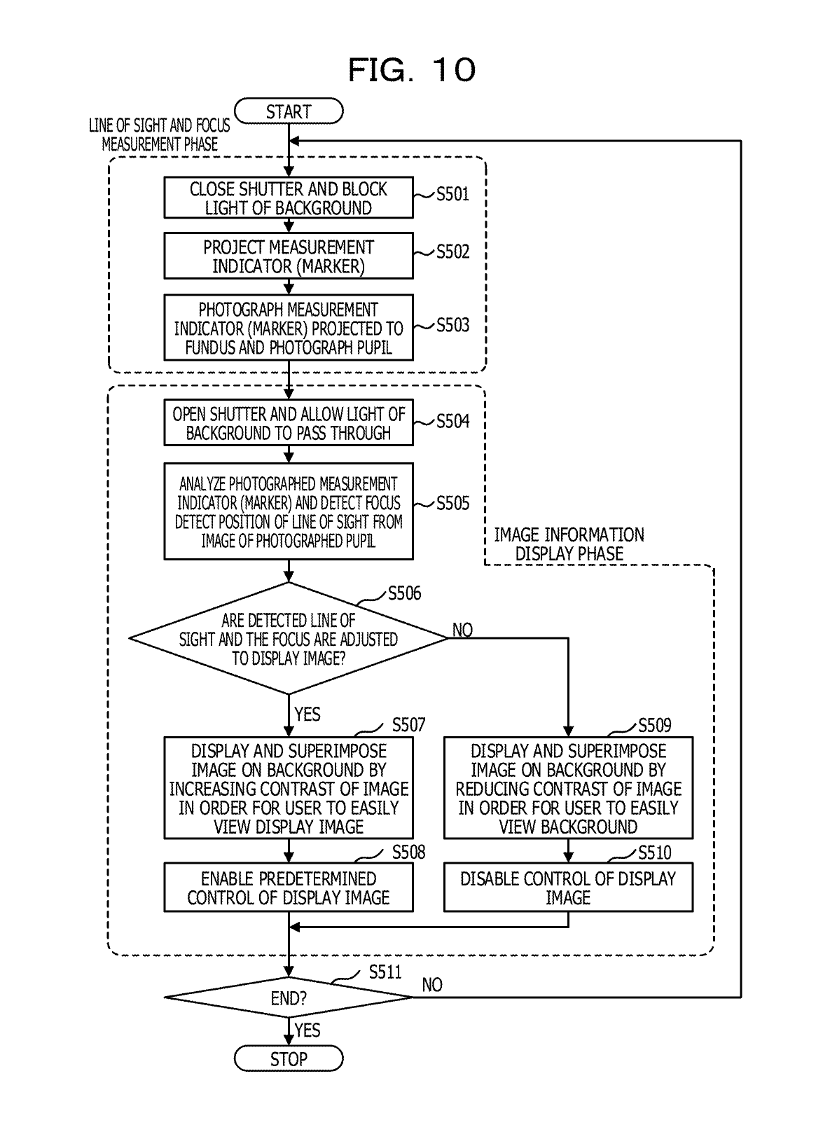

FIG. 10 illustrates an example of a control process. The CPU 122 reads a program from the ROM 124 into the RAM 123 and executes the program to perform the following control process.

Operation S501, Operation S502, and Operation S503 may correspond to the line of sight and focus measurement phase 401 illustrated in FIG. 9. Operation S504 to Operation S510 may correspond to the image information display phase illustrated in FIG. 9.

The CPU 122 closes the shutter 131 at Operation S501. Therefore, the light (extraneous light) entering the eye from the external world is blocked. When the projector 125 is irradiating the display image, the CPU 122 instructs the projector 125 to stop the irradiation of the display image, and the projector 125 stops the irradiation of the display image.

The projector 125 irradiates the marker onto the eye to be examined based on the instruction of the CPU 122 at Operation S502.

The camera 126 photographs the marker projected to the fundus of the eye to be examined based on the instruction of the CPU 122, and the CPU 122 stores the photographed image 201 in the RAM 123 at Operation S503. The camera 126 photographs the pupil to be examined based on the instruction of the CPU 122 and the CPU 122 stores the photographed image 211 in the RAM 123.

Photographing of the pupil to be examined may be performed between Operation S504 and Operation S505 rather than at Operation S503. For example, the photographing of the pupil to be examined may be performed when the shutter 131 is opened.

The CPU 122 opens the shutter 131 at Operation S504. The CPU 122 analyzes the photographed image 201 and detects the focus at Operation S505. The CPU 122 calculates the focus based on the magnitude of the photographed marker. The CPU 122 analyzes the photographed image 211 to detect the position of the line of sight.

The CPU 122 determines whether the detected line of sight and the focus are adjusted or not to an image (display image) displayed on the half mirror 141 by the projector 125 at Operation S506. For example, it is determined whether the user gazes the display image or not. For example, when the detected line of sight and the focus are fallen within a range, respectively, the CPU 122 determines that the line of sight and the focus are adjusted to the display image.

When it is determined that the detected line of sight and the focus are adjusted to the display image, the control process proceeds to Operation S507 and otherwise, proceeds to Operation S509.

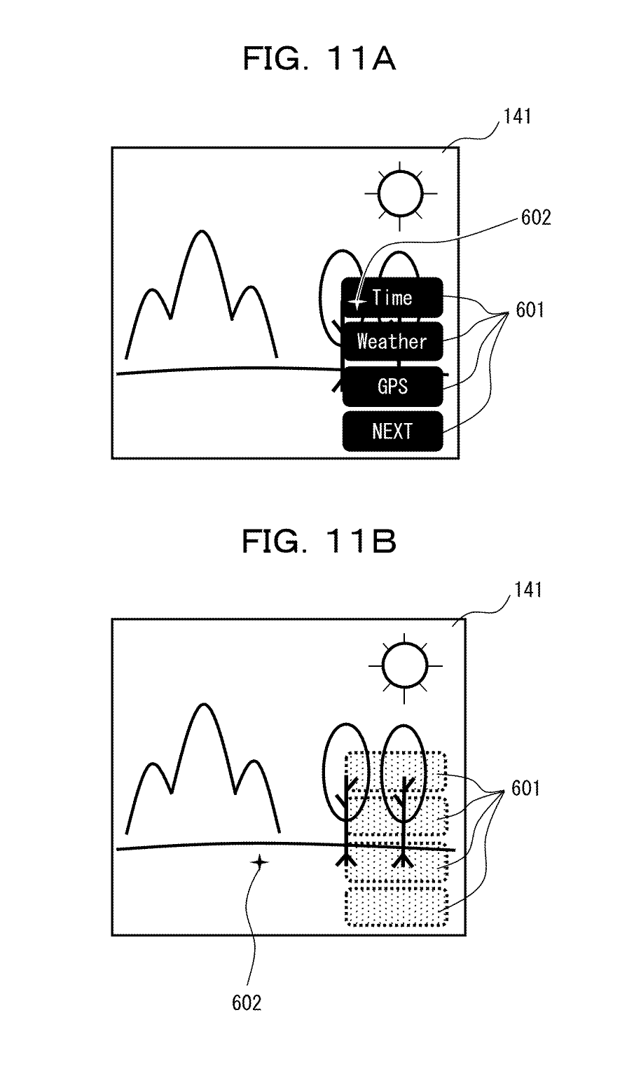

The projector 125 irradiates the display image based on the instruction of the CPU 122 at Operation S507. The CPU 122 increases the contrast of the display image irradiated by the projector 125. FIG. 11A and FIG. 11B illustrate an example of a display image. Therefore, as illustrated in FIG. 11A, the contrast of the display image 601 such as a menu projected to the half mirror 141 is increased and the user becomes able to easily view the display image 601. In FIG. 11A, the menu which includes “Time”, “Weather”, “GPS”, and “NEXT” is displayed as the display image in the half mirror 141. The CPU 122 causes the projector 125 to irradiate an image of a cross-shaped cursor 602 indicating the position of the line of sight of user. The CPU 122 may cause the shape or the brightness of the cursor 602 to be varied according to the detected focus.

The CPU 122 enables the control corresponded to the display image 601 at Operation S508. For example, when the position of the line of sight and focus of the user are adjusted to a display image 601, the CPU 122 causes an operation from the user to be able to be received and performs the processing such as a selection of the menu or a scrolling for the display image 601 according to the operation. For example, when the user has adjusted the line of sight and the focus to “Time” of the menu illustrated in FIG. 11A, the CPU 122 becomes able to receive the instruction to perform the processing. When an operation button installed in the HMD device 101 is depressed in a state where the user has adjusted the line of sight and the focus to the “Time”, the CPU 122 receives the instruction to perform the processing. The CPU 122 determines that the “Time” is selected from the line of sight and the focus of the user, and displays a time on the half mirror 141 by an irradiation of an image of the time from the projector 125.

As described above, only when the detected line of sight and focus have been adjusted to the display image 601, the CPU 122 receives the instruction to perform the processing and performs the processing. For example, only when the detected line of sight and focus have been adjusted to the display image 601, the CPU 122 may be able to perform the processing corresponded to the display image 601.

The projector 125 irradiates the display image based on the instruction of the CPU 122 at Operation S509. The CPU 122 causes the contrast of the display image irradiated by the projector 125 to be reduced. Therefore, as illustrated in FIG. 11B, the contrast of the display image 601 such as the menu projected to the half mirror 141 is reduced and this reduction of the contrast makes it easy for the user to view the external world (background) being superimposed with the display image 60.

The CPU 122 disables the control corresponded to the display image 601 at Operation S509. For example, the CPU 122 disables the operation such as the selection of the menu or the scrolling.

When terminating a processing at Operation S511, the control process is ended and otherwise, when not terminating the processing at Operation S511, the control process goes back to Operation S501.

In the HMD device described above, since the extraneous light including strong infrared light is blocked at the time when the marker is photographed, a disturbance (noise) is reduced and the image of the photographed marker becomes clear such that a measurement accuracy of the focus may be improved.

In the HMD device described above, since the disturbance is reduced, the focus of the user may be detected with the infrared ray of small power.

In the HMD device described above, since the control is performed based on the position of the line of sight and the focus, the possibility of an occurrence of unintended operations of the user may be reduced.

All examples and conditional language recited herein are intended for pedagogical purposes to aid the reader in understanding the invention and the concepts contributed by the inventor to furthering the art, and are to be construed as being without limitation to such specifically recited examples and conditions, nor does the organization of such examples in the specification relate to an illustrating of the superiority and inferiority of the invention. Although the embodiments of the present invention have been described in detail, it should be understood that the various changes, substitutions, and alterations could be made hereto without departing from the spirit and scope of the invention.

What is claimed is:

1. An information processing apparatus comprising:
a shutter configured to block extraneous light which enters into an eye;
an irradiation unit configured to irradiate a marker to the eye by infrared light;
a photographing unit configured to photograph the marker which is projected onto a fundus of the eye to be examined when the shutter is closed; and
a processing unit configured to detect a focus based on an image of the marker photographed by the photographing unit.

2. The information processing apparatus according to claim 1, further comprising:
a light entrance part; and
a frame configured to support one of the shutter, the irradiation unit, the photographing unit, and the processing unit,
wherein the shutter is configured to block the extraneous light entering into the eye from the light entrance part.

3. The information processing apparatus according to claim 1, wherein the shutter is closed regularly.

4. The information processing apparatus according to claim 1, wherein the irradiation unit is configured to irradiate the marker when the shutter is closed.

5. The information processing apparatus according to claim 1, further comprising:
a display unit configured to reflect a display image by allowing the extraneous light to pass through,
wherein the photographing unit is configured to photograph a pupil of the eye,
the irradiation unit is configured to irradiate the display image when the shutter is opened, and
the processing unit is configured to detect a position of a line of sight based on an image of the pupil photographed by the photographing unit.

6. The information processing apparatus according to claim 5, wherein a control corresponded to the display image is enabled when the line of sight and the focus are adjusted to the display image.

7. The information processing apparatus according to claim 5, wherein the processing unit is configured to increase a contrast of the display image when the line of sight and the focus are adjusted to the display image.

8. A focus detection method comprising:
closing a shutter configured to block extraneous light which enters into an eye;
irradiating a marker to the eye by infrared light;
photographing the marker which is projected onto a fundus of the eye when the shutter is closed; and
detecting a focus based on an image of the marker photographed by the photographing unit.

9. The focus detection method according to claim 8, wherein the shutter blocks the extraneous light entering into the eye from a light entrance part.

10. The focus detection method according to claim 8, wherein the shutter is closed regularly.

11. The focus detection method according to claim 8, wherein the irradiating is performed when the shutter is closed.

12. The focus detection method according to claim 8, further comprising:
reflecting a display image by allowing the extraneous light to pass through;
photographing a pupil of the eye; and
detecting a position of a line of sight based on an image of the pupil photographed by the photographing unit.

13. The focus detection method according to claim 12, further comprising:
enabling a control corresponded to the display image when the line of sight and the focus are adjusted to the display image.

14. The focus detection method according to claim 12, further comprising:
increasing a contrast of the display image when the line of sight and the focus are adjusted to the display image.

15. An information processing system comprising:
a processor configured to execute a program; and
a memory configured to store the program,
wherein the processor, based on the program, is configured to,
close a shutter configured to block extraneous light which enters into an eye;
irradiate a marker to the eye by infrared light;
photograph the marker projected onto a fundus of the eye when the shutter is closed; and
detect a focus based on an image of the marker photographed by the photographing unit.

16. The information processing system according to claim 15, further comprising:
a light entrance part; and
a frame configured to support one of the shutter, the irradiation unit, the photographing unit, and the processing unit,
wherein the shutter is configured to block the extraneous light entering into the eye from the light entrance part.

17. The information processing system according to claim 15, wherein the shutter is closed regularly.

18. The information processing system according to claim 15, wherein the irradiation unit is configured to irradiate the marker when the shutter is closed.

19. The information processing system according to claim 15, further comprising:
a display unit configured to reflect a display image by allowing the extraneous light to pass through,
wherein the photographing unit is configured to photograph a pupil of the eye,
the irradiation unit is configured to irradiate the display image when the shutter is opened, and
the processing unit is configured to detect a position of a line of sight based on an image of the pupil photographed by the photographing unit.

20. The information processing system according to claim 19, wherein a control corresponded to the display image is enabled when the line of sight and the focus are adjusted to the display image.
